(19) United States
(12) Patent Application Publication
(10) Pub. No.: US 2020/0352509 A1
(43) Pub. Date: Nov. 12, 2020

(54) BODY-ATTACHABLE DETECTION DEVICE

(71) Applicant: SHARP KABUSHIKI KAISHA, Sakai City, Osaka (JP)

(72) Inventors: MOTOKI TANIMURA, Sakai City, Osaka (JP); SUMIO HORIMOTO, Sakai City, Osaka (JP)

(21) Appl. No.: 16/642,235

(22) PCT Filed: Dec. 8, 2017
(86) PCT No.: PCT/JP2017/044203

§ 371 Date: Feb. 26, 2020

(30) Foreign Application Priority Data
Aug. 30, 2017 (JP) 2017-165945

(51) Int. Cl. A61B 5/00; A61B 5/11

(52) U.S. Cl. CPC A61B 5/6815; A61B 5/11; A61B 5/486; A61B 5/4205

(57) ABSTRACT

A body-attachable detection device includes a light-emitting unit; a photoelectric conversion unit; a control unit; and an attachment portion, wherein the attachment portion is provided to attach the light-emitting unit, the photoelectric conversion unit, and the control unit to a living body, the light-emitting unit is configured to project light onto a target part from which movements of the living body are to be detected, the photoelectric conversion unit is configured to receive light reflected by the target part and to generate photocurrent, and the control unit is configured to adjust, in accordance with the photocurrent generated in the photoelectric conversion unit, an amount of light to be projected onto the target part and to detect movements of the target part in accordance with changes in photocurrent that occur in the photoelectric conversion unit after the amount of light is adjusted.

### TECHNICAL FIELD

The present invent relates to a body-attachable detection device.

### BACKGROUND ART

Attention is being given to wearable sensors from the viewpoint of, for example, lifestyle improvement, early detection of diseases, and physical condition management.

For example, wearable sensors capable of counting chewing strokes are known (see, for example, PTL 1 and PTL 2). Such a sensor counts chewing strokes in such a manner as to detect movements of a jaw by using a distance sensor that measures the distance between the sensor and the jaw.

Eating food without sufficient chewing may cause obesity or indigestion. The wearable sensor indicates the number of chewing strokes in a numerical form such that the user can easily check the number of chewing strokes and is prompted to eat food with enough chewing. The wearable sensor also enables control of the number of chewing strokes and is thus useful for health care management.

### CITATION LIST

### Patent Literature

PTL 1: Japanese Unexamined Patent Application Publication No. 2015-208633

PTL 2: Japanese Unexamined Patent Application Publication No. 2015-208634

### SUMMARY OF INVENTION

### Technical Problem

A conventional wearable sensor that counts chewing strokes includes a distance sensor whose specifications are fixed. When the sensor measures the distance between the sensor and a jaw, the accuracy of detecting movements of the jaw may suffer due to, for example, the state in which the wearable sensor is attached, the skin color, the skin condition, lighting (incandescent lamps, fluorescent lamps, or LEDs), or the location (indoor or outdoor).

The present invention therefore has been made in view of such circumstances and provides a body-attachable detection device whose sensitivity to movements of a target part remains good irrespective of possible changes in, for example, the attachment state, the color of the skin on the target part, the amount of moisture in the skin, the quality of the skin, and the shape of the target part.

### Solution to Problem

The present invention provides a body-attachable detection device including a light-emitting unit, a photoelectric conversion unit, a control unit, and an attachment portion. The attachment portion is provided to attach the light-emitting unit, the photoelectric conversion unit, and the control unit to a living body. The light-emitting unit is configured to project light onto a target part from which movements of the living body are to be detected. The photoelectric conversion unit is configured to receive light reflected by the target part and to generate photocurrent. The control unit is configured to adjust, in accordance with the photocurrent generated in the photoelectric conversion unit, the amount of light to be projected onto the target part and to detect movements of the target part in accordance with changes in photocurrent that occur in the photoelectric conversion unit after the amount of light is adjusted.

### Advantageous Effects of Invention

The light-emitting unit is configured to project light onto the target part from which movements of the living body are to be detected. The photoelectric conversion unit is configured to receive light reflected by the target part and to generate photocurrent. Thus, movements of the target part cause changes in the amount of light projected onto the target part and reflected toward the photoelectric conversion unit. The photocurrent generated in the photoelectric conversion unit changes in magnitude accordingly.

The control unit is configured to adjust, in accordance with the photocurrent generated in the photoelectric conversion unit, the amount of light to be projected onto the target part. Thus, the target part may be irradiated with an appropriate amount of light in accordance with, for example, the state in which the body-attachable detection device is attached, the color of the skin on the target part, and the condition of the skin.

The control unit is configured to detect movements of the target part in accordance with changes in photocurrent that occur in the photoelectric conversion unit after the amount of light is adjusted. Thus, movements of the target part may be optimally detected in a manner suited to the attachment state and the skin condition without being affected by uncertain factors such as the attachment state, the color of the skin on the target part, the amount of moisture in the skin, the quality of the skin, and the shape of the target part. The sensitivity to movements of the target part thus remains good irrespective of possible changes in, for example, the attachment state, the color of the skin on the target part, the amount of moisture in the skin, the quality of the skin, and the shape of the target part.

### BRIEF DESCRIPTION OF DRAWINGS

FIG. 1 is a schematic perspective view of a body-attachable detection device according to an embodiment of the present invention.

FIG. 2 schematically illustrates the state in which the body-attachable detection device according to an embodiment of the present invention is attached.

FIG. 3 is a schematic sectional view of the body-attachable detection device according to an embodiment of the present invention.

FIGS. 4(a) and 4(b) illustrate how an optical path changes when the distance between a target part and each of a light-emitting unit and a photoelectric conversion unit changes.

FIG. 5 illustrates the waveform of chewing detected by the body-attachable detection device according to an embodiment of the present invention.

FIG. 6 is a flowchart illustrating optimization processing executed by the body-attachable detection device according to an embodiment of the present invention.

FIG. 7 is a flowchart of counting and measurement processing executed by the body-attachable detection device according to an embodiment of the present invention.

FIGS. 8(a) and 8(b) illustrate optical paths that are formed when movements of the target part are detected by using the body-attachable detection device according to an embodiment of the present invention.

FIG. 9 is a flowchart illustrating optimization processing executed by the body-attachable detection device according to an embodiment of the present invention.

FIG. 10 is a schematic enlarged view of a region A enclosed by the broken line in FIG. 3.

FIG. 11 is a schematic exploded view of the body-attachable detection device according to an embodiment of the present invention.

FIG. 12(a) is a schematic perspective view of a holder, and FIG. 12(b) is a schematic side view of the holder.

FIG. 13 is a graph illustrating experimental results.

FIG. 14 is a graph illustrating experimental results.

### DESCRIPTION OF EMBODIMENTS

A body-attachable detection device according to the present invention includes a light-emitting unit, a photoelectric conversion unit, a control unit, and an attachment portion. The attachment portion is provided to attach the light-emitting unit, the photoelectric conversion unit, and the control unit to a living body. The light-emitting unit is configured to project light onto a target part from which movements of the living body are to be detected. The photoelectric conversion unit is configured to receive light reflected by the target part and to generate photocurrent. The control unit is configured to adjust, in accordance with the photocurrent generated in the photoelectric conversion unit, the amount of light to be projected onto the target part and to detect movements of the target part in accordance with changes in photocurrent that occur in the photoelectric conversion unit after the amount of light is adjusted.

The light-emitting unit preferably includes a light-emitting diode. The control unit is preferably configured to adjust the amount of light to be projected onto the target part by changing the magnitude of voltage applied across the light-emitting diode, by changing the magnitude of current flowing through the light-emitting diode, or by changing the period of time over which current flows through the light-emitting diode. Owing to this configuration, the amount of light to be projected onto the target part may be easily adjusted.

The detection device according to the present embodiment preferably includes at least one neutral-density filter. The neutral-density filter and the control unit are preferably provided in such a manner that the amount of light to be projected onto the target part is adjusted by inserting the neutral-density filter into a light projection path between the light-emitting unit and the target part and by replacing the neutral-density filter placed in the light projection path between the light-emitting unit and the target part with another neutral-density filter. Owing to this configuration, the amount of light to be projected onto the target part may be easily adjusted.

It is preferred that the detection device according to the present invention further include a dimming structure placed in the light projection path between the light-emitting unit and the target part. The dimming structure and the control unit are preferably provided in such a manner that the amount of light to be projected onto the target part is adjusted through the dimming structure. Owing to this configuration, the amount of light to be projected onto the target part may be easily adjusted.

The control unit is preferably configured to: determine changes in photocurrent that occur in the photoelectric conversion unit due to movements of the target part while the target part is irradiated with a first amount of light; determine changes in photocurrent that occur in the photoelectric conversion unit due to movements of the target part while the target part is irradiated with light in an amount different from the first amount; select, from the amounts of light projected onto the target part, the amount of light effecting the most significant changes in photocurrent; and adjust the amount of light to be projected onto the target part so that the target part is irradiated with the selected amount of light. Owing to this configuration, the amount of light to be projected onto the target part may be easily adjusted in such a manner as to achieve a high degree of sensitivity.

The attachment portion preferably includes: a housing that accommodates the light-emitting unit, the photoelectric conversion unit, and the control unit; an ear hook portion; and a holder fixed to the housing and made of metal. The ear hook portion preferably includes a core portion made of metal and a rubber portion that covers the core portion. It is preferred that the ear hook portion be removably fastened to the housing in such a manner that a protruding portion provided on one of the holder and the core portion fits in a recessed portion or an opening provided in the other one of the holder and the core portion. The protruding portion is preferably a hemispherical projection. The detection device may be thus attached to the ear with a comfortable fit. Furthermore, the detection device is less prone to damage, which could be otherwise caused by attachment or removal of the ear hook portion. When the protruding portion fits into the recessed portion or the opening, the user can feel a click and can thus recognize that the core portion is in the right place in the holder.

The following describes the present invention in more detail by referring to embodiments. Configurations in the accompanying drawings or in the following description are merely examples. The scope of the present invention is not limited to the configurations in the drawings or in the following description.

### First Embodiment

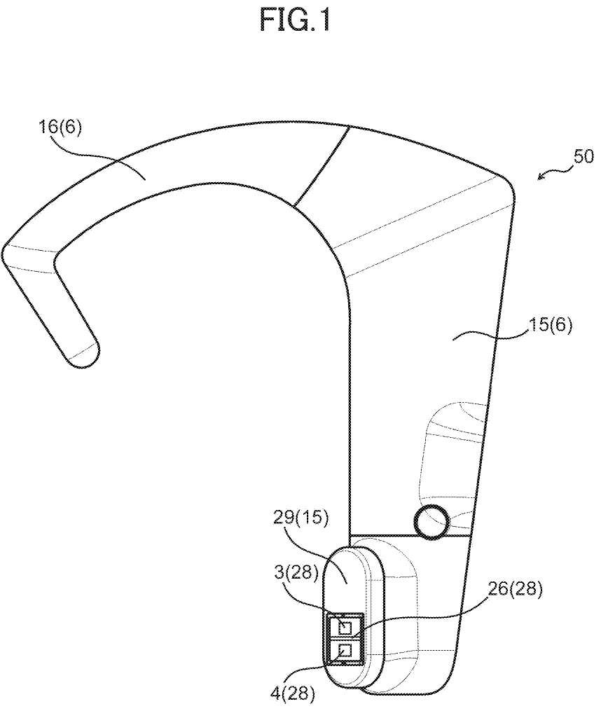

FIGS. 1 to 6 illustrate a body-attachable detection device according to a first embodiment. Description of these drawings is similar to the above.

A body-attachable detection device 50 according to the first embodiment includes a light-emitting unit 3, a photoelectric conversion unit 4, a control unit 5, and an attachment portion 6. The attachment portion 6 is provided to attach the light-emitting unit 3, the photoelectric conversion unit 4, and the control unit 5 to a living body. The light-emitting unit 3 is configured to project light onto a target part 8, from which movements of the living body are to be detected. The photoelectric conversion unit 4 is configured to receive light reflected by the target part 8 and to generate photocurrent. The control unit 5 is configured to adjust, in accordance with the photocurrent generated in the photoelectric conversion unit 4, the amount of light to be projected onto the target part 8 and to detect movements of the target part 8 in accordance with changes in photoelectric current that occur in the photoelectric conversion unit 4 after the amount of light is adjusted.

The body-attachable detection device 50 is a wearable device and detects movements of a living body. Movements of a living body that are to be detected by the detection device 50 are, for example, movements of a jaw, muscles, an arm, or a leg. The movements may be detected in such a manner that the detection device 50 detects movements of, for example, the skin or the clothes on the part concerned (the target part 8).

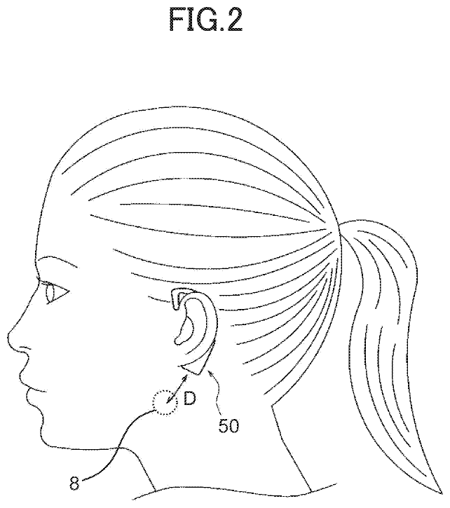

The target part 8 is the part from which movements of the living body are to be detected. Movements of the living body are detected in such a manner that the detection device 50 detects movements of the target part 8. When, for example, the detection device 50 is configured to detect chewing, the detection device 50 detects chewing by detecting movements of the skin on a lower jaw located below an ear. As illustrated in FIG. 2, the detection device 50 illustrated in FIG. 1 may be attached to the ear such that movements of the skin on the lower jaw may be detected.

Movements of a living body that are to be detected by the detection device 50 may be, for example, movements of an inner part of the body. The detection device 50 can, for example, detect pulsations of a blood vessel. In this case, the blood vessel or blood flowing through the blood vessel is regarded as the target part 8.

The attachment portion 6 is provided to attach the light-emitting unit 3, the photoelectric conversion unit 4, and the control unit 5 to a living body. In this way, the detection device 50 can be attached to the body to detect movements of the target part 8 continuously.

The attachment portion 6 may, for example, include: a housing 15, which accommodates the light-emitting unit 3, the photoelectric conversion unit 4, and the control unit 5; and a portion via which the housing 15 can be attached to the living body. As illustrated in FIGS. 1 and 2, the attachment portion 6 may, for example, include the housing 15 and an ear hook portion 16 so that the detection device 50 can be attached to an ear. The attachment portion 6 may include the housing 15 and a band so that the detection device 50 can be attached to an arm. The attachment portion 6 may include the housing 15 and a sticky portion so that the detection device 50 can be stuck on, for example, the skin.

The control unit 5 controls, for example, the light-emitting unit 3 and the photoelectric conversion unit 4. The control unit 5 may include, for example, a calculation unit, a storage unit, an input unit, an output unit, a communication unit, a light emission control unit, and a power supply circuit. The control unit 5 may include, for example, a control board.

The light-emitting unit 3 is configured to project light onto the target part 8, from which movements of the living body are to be detected. Light to be emitted by the light-emitting unit 3 is not limited and may be any type of light whose reflection can be detected by the photoelectric conversion unit 4. Such light is, for example, infrared radiation, red light, green light, or blue light.

The light-emitting unit 3 may include a light emitter and a lens. The light emitter is, for example, a light-emitting diode (LED), a laser element, or an electroluminescent element.

It is preferred that the light emitter included in the light-emitting unit 3 be a light-emitting diode. Due to, for example, changing of the magnitude of voltage applied across the light-emitting diode, changing of the magnitude of current flowing through the light-emitting diode, or changing of the period of time over which current flows through the light-emitting diode, the luminance of the light-emitting unit 3 changes and the amount of light to be projected onto the target part 8 is adjusted accordingly.

Owing to the lens included in the light-emitting unit 3, the target part 8 may be efficiently irradiated with light.

The photoelectric conversion unit 4 is configured to receive light emitted by the light-emitting unit 3 and reflected by the target part 8 and to generate photocurrent. The photoelectric conversion unit 4 may include, for example, a phototransistor, a photodiode, or a photocell. The photoelectric conversion unit 4 may also include a lens. This configuration enables efficient photoelectric conversion. The lens may include a filter. For example, the lens of the photoelectric conversion unit 4 may include a visible-light blocking filter in a case where the light-emitting unit 3 is configured to irradiate the target part 8 with infrared light and the photoelectric conversion unit 4 is configured to receive light reflected by the target part 8. This eliminates or reduces the possibility that photoelectric conversion in the photoelectric conversion unit 4 will be affected by, for example, illumination light beams. The detection sensitivity of the detection device 50 may be enhanced accordingly.

The light-emitting unit 3 and the photoelectric conversion unit 4 may be disposed side by side. This layout facilitates the entry of light emitted by the light-emitting unit 3 and reflected by the target part 8 into the photoelectric conversion unit 4.

A partition 26 may be disposed between the light-emitting unit 3 and the photoelectric conversion unit 4. This suppresses the direct entry of light emitted by the light-emitting unit 3 into the photoelectric conversion unit 4.

The detection device 50 may include spacers 29 facing each other across both the projection path of light projected by the light-emitting unit 3 onto the target part 8 and the reflection path of light reflected by the target part 8 and incident on the photoelectric conversion unit 4. In this case, the light-emitting unit 3 and the photoelectric conversion unit 4 may not be placed too close to the target part 8, and movements of the target part 8 may be thus detected with a high degree of sensitivity.

Light emitted by the light-emitting unit 3 is projected onto the target part 8 as illustrated in FIG. 4(a). Light projected onto the target part 8 may be transmitted through the target part 8, may be scattered at the target part 8, or may be reflected by the target part 8. Part of light reflected by the target part 8 enters the photoelectric conversion unit 4, where photocurrent is generated via photoelectric conversion. Along with the movement of the target part 8 from the state in 4(a) to the state in 4(b), the distance between the target part 8 and each of the light-emitting unit 3 and the photoelectric conversion unit 4 changes from D₁ to D₂. The angle at which light is incident on the target part 8 changes accordingly. This makes changes in transmission, scattering, and reflection of light at the target part 8. As a result, the amount of light incident on the photoelectric conversion unit 4 changes, and the photocurrent generated in the photoelectric conversion unit 4 changes in magnitude accordingly.

When the detection device 50 is attached to an ear as illustrated in FIG. 2 so that chewing is detected from movements of a jaw (the target part 8), the distance D between the target part 8 and each of the light-emitting unit 3 and the photoelectric conversion unit 4 cyclically shifts, due to chewing, back and forth between the state illustrated in FIG. 4(a) and the state illustrated in FIG. 4(b). Similarly, the angle at which light is incident on the target part 8 cyclically changes. As a result, the photocurrent generated in the photoelectric conversion unit 4 cyclically changes in magnitude. The photocurrent generated in the photoelectric conversion unit 4 changes in magnitude, for example, as illustrated in FIG. 5. The number of chewing strokes may be determined by counting cycles of the continuous change in the magnitude of photocurrent. The greater the continuous change in the magnitude of photocurrent is, the higher the detection sensitivity for the number of chewing stroke is. The smaller the change in the magnitude of photocurrent is, the lower the detection sensitivity is.

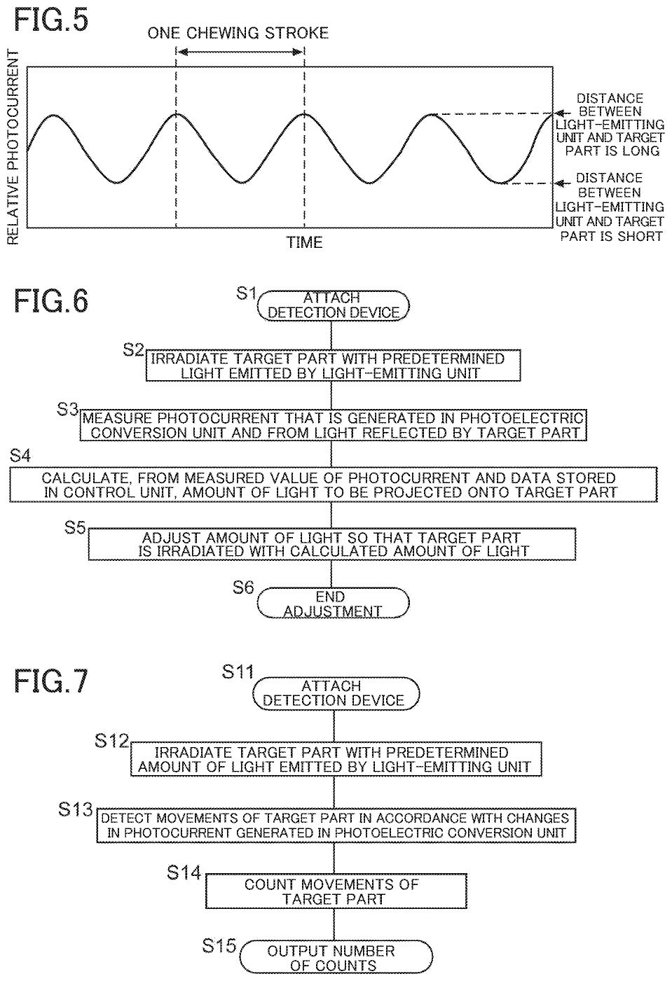

The detection sensitivity of the detection device 50 may vary depending on, for example, the state in which the detection device 50 is attached, the color of the skin on the target part 8, the amount of moisture in the skin, the quality of the skin, the way in which the target part 8 moves, the lighting (incandescent lamps, fluorescent lamps, or LEDs), or the location (indoor or outdoor). In the present embodiment, the amount of light to be projected onto the target part 8 is adjusted (in optimization processing) by, for example, the control unit 5 in accordance with the photocurrent generated in the photoelectric conversion unit 4 before the processing for detecting movements of the target part 8 is started. The amount of light may be adjusted, for example, by following the flowchart in FIG. 6.

For example, the detection device 50 is attached to a living body in such a manner as to irradiate the target part 8 with light (Step S1), and the target part 8 is irradiated with a predetermined amount of light emitted by the light-emitting unit 3 (Step S2). The amount of light that is conceivably most favorable for the detection of movements of the target part 8 may be given as the predetermined amount of light. The magnitude of the photocurrent generated in the photoelectric conversion unit 4 by using light projected on and reflected by the target part 8 is measured (Step S3).

Subsequently, the amount of light to be projected onto the target part 8 is calculated from the measured value of photocurrent and data stored in the control unit 8 (Step S4). The data is prestored in the control unit and describes the relationship between the measured value of photocurrent and the amount of irradiation with which a high degree of detection sensitivity is achieved. The data may describe the relationship between the measured value of photocurrent and the luminance of the light-emitting unit 3 (or the corresponding voltage value, the corresponding current value, and the corresponding period of time over which current flows).

The amount of light is then adjusted so that the target part 8 is irradiated with the calculated amount of light (Step S5). When the light-emitting unit 3 includes a light-emitting diode, the luminance of the light-emitting unit 3 changes due to, for example, changing of the magnitude of voltage applied across the light-emitting diode, changing of the magnitude of current flowing through the light-emitting diode, or changing of the period of time over which current flows through the light-emitting diode. The amount of light to be projected onto the target part 8 is adjusted accordingly.

For example, a variable resistor may be connected in series with the light-emitting diode. Adjusting the resistance value of the variable resistor enables adjustment of current flowing through the light-emitting diode or adjustment of voltage applied across the light-emitting diode, and the luminance of the light-emitting diode is adjusted accordingly. When the light-emitting diode is subjected to, for example, PWM control, changing the pulse width to adjust the ratio of ON time (duty ratio) enables adjustment of the luminance of the light-emitting diode.

The counting and measuring processing may be then executed by following the flowchart in FIG. 7. For example, while the target part 8 is irradiated with the adjusted amount of light, photocurrent generated in the photoelectric conversion unit 4 is measured and movements of the target part 8 are detected in accordance with changes in photocurrent (Steps S12 and S13). Detecting movements of the target part 8 after adjusting the amount of light will enable detection of movements of the target part 8 with a high degree of sensitivity irrespective of possible changes in the state in which the detection device 50 is attached or possible changes in the state of the target part 8.

The movements of the target part 8 are counted (Step S14), and the number of counts is output to, for example, a monitor (Step S15). The monitor may be included in the detection device 50 or wirelessly connected to the detection device 50 or may be a mobile terminal such as a smartphone, a tablet PC, or a notebook computer. The number of counts may be output to, for example, a personal computer so that the number of chewing strokes may be stored in the personal computer.

The optimization processing and the counting and measurement processing, which have been described as separate flows, may be serial processing.

### Second Embodiment

In a second embodiment, the optimization processing includes: calculating the amount of light to be projected onto the target part 8; and adjusting, through a neutral-density filter 10, the amount of light to be projected onto the target part 8. The configuration is otherwise identical to the configuration in the first embodiment. Description of the first embodiment holds true for the second embodiment as long as no inconsistencies are produced.

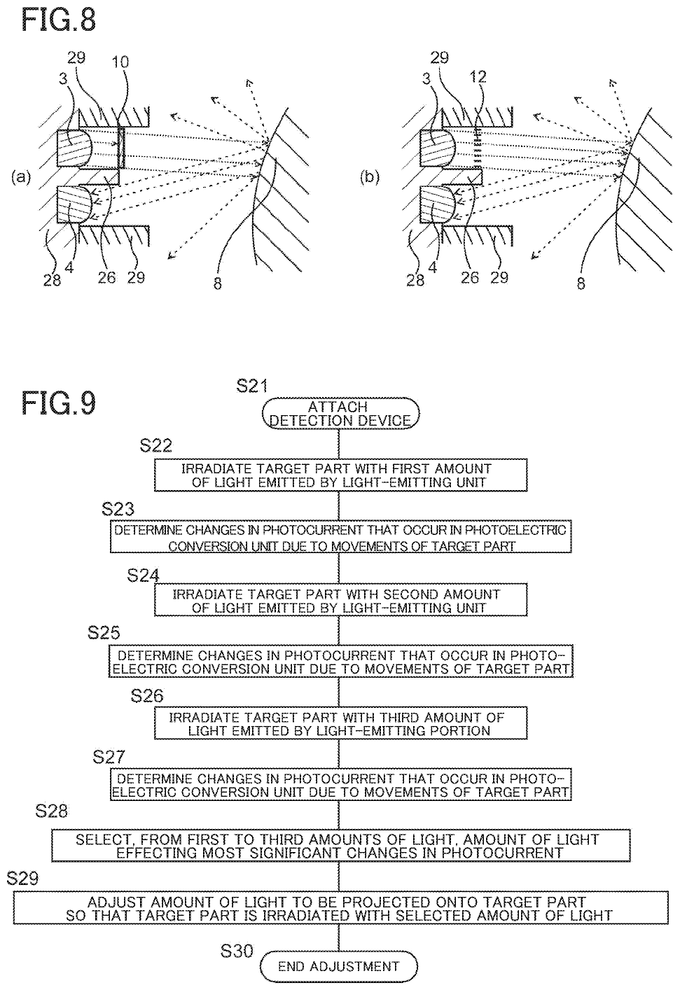

The detection device 50 may include the neutral-density filter 10, which may be placed in a light projection path between the light-emitting unit 3 and the target part 8. The neutral-density filter 10 may be inserted into the light projection path between the light-emitting unit 3 and the target part 8 and may be withdrawn from the light projection path. Owing to this configuration, the target part 8 may be irradiated with light that is not transmitted through the neutral-density filter 10 or with light that is transmitted through the neutral-density filter 10. The amount of light to be projected onto the target part 8 is thus adjustable. Insertion and withdrawal of the neutral-density filter may be controlled by the control unit 8. The neutral-density filter 10 may be disposed, for example, as illustrated in FIG. 8(a).

The detection device 50 may include a plurality of neutral-density filters 10, which may be placed in the light projection path between the light-emitting unit 3 and the target part 8. The neutral-density filters 10 may be individually inserted into the light projection path and withdrawn from the light projection path. The neutral-density filters 10 may have different transmittances. Owing to this configuration, the amount of light to be projected onto the target part 8 is adjustable through the use of the neutral-density filters 10.

### Third Embodiment

In a third embodiment, the optimization processing includes: calculating the amount of light to be projected onto the target part 8; and adjusting, through a dimming structure 12, the amount of light to be projected onto the target part 8. The configuration is otherwise identical to the configuration in the first embodiment. Description of the first embodiment holds true for the third embodiment as long as no inconsistencies are produced.

The detection device 50 may include the dimming structure 12, which may be placed in the light projection path between the light-emitting unit 3 and the target part 8. The dimming structure 12 is not limited and may be any structure capable of dimming light projected onto the target part 8. Such a structure is, for example, a movable light-shielding plate. When the light-shielding plate is placed parallel to the light projection path, light emitted by the light-emitting unit 3 is mostly projected onto the target part 8. When the light-shielding plate is placed oblique to the light projection path, light emitted by the light-emitting unit 3 is partially shielded by the shielding plate, and the target part 8 is thus irradiated with a reduced amount of light. Thus, the amount of light to be projected onto the target part 8 may be adjusted in accordance with the inclination of the light-shielding plate. The dimming effected through the dimming structure 12 may be controlled by the control unit 5. The dimming structure 12 may be disposed, for example, as illustrated in FIG. 8(b).

### Fourth Embodiment

In a fourth embodiment, the optimization processing includes: determining changes in photocurrent that occur in the photoelectric conversion unit 4 due to movements of the target part 8 while the target part 8 is irradiated with a first amount of light; determining changes in photocurrent that occur in the photoelectric conversion unit 4 due to movements of the target part 8 while the target part 8 is irradiated with light in an amount different from the first amount; selecting, from the amounts of light projected onto the target part 8, the amount of light effecting the most significant changes in photocurrent; and adjusting the amount of light to be projected onto the target part 8 so that the target part 8 is irradiated with the selected amount of light. The configuration is otherwise identical to the configuration in the first to third embodiments. Description of the first to third embodiments holds true for the fourth embodiment as long as no inconsistencies are produced.

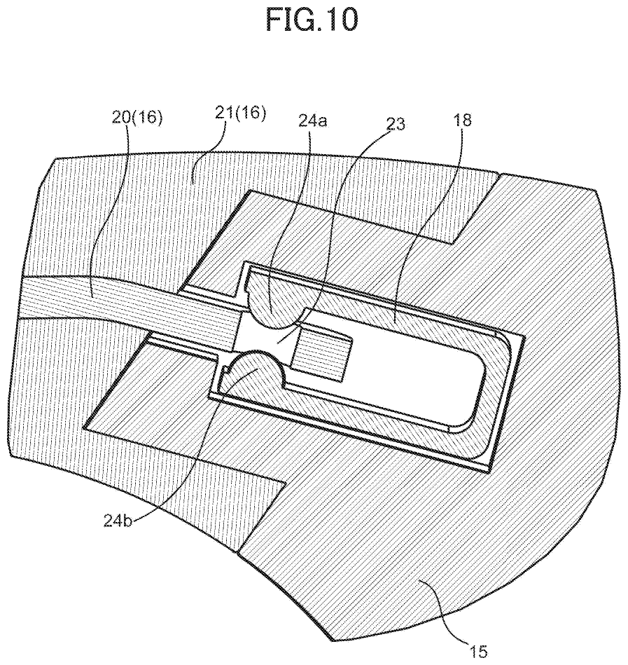

In the present embodiment, the amount of light may be adjusted, for example, by following the flowchart in FIG. 9.

For example, the detection device 50 is attached to a living body in such a manner as to irradiate the target part 8 with light (Step S21). While the target part 8 is irradiated with the first amount of light emitted by the light-emitting unit 3, changes in photocurrent that occur in the photoelectric conversion unit 4 due to movements of the target part 8 are determined (Steps S22 and S23).

Subsequently, the amount of light to be projected onto the target part 8 is set to a second amount, and changes in photocurrent that occur in the photoelectric conversion unit 4 due to movements of the target part 8 are determined (Steps S24 and S25).

Changes in photocurrent may be determined two, three, four, five, six, seven, or eight times, each with a different amount of light (Steps S26 and S27).

Subsequently, the amount of light effecting the most significant changes in photocurrent is selected from the amounts of light projected onto the target part 8 (Step 28). The amount of light to be projected onto the target part 8 is then adjusted so that the target part 8 is irradiated with the selected amount of light (Step S29). The amount of light may be adjusted by controlling current flowing through the light-emitting diode as in the first embodiment, by using the neutral-density filter 10 as in the second embodiment, or by using the dimming structure 12 as in the third embodiment.

The counting and measuring processing may be then executed by following the flowchart in FIG. 7. For example, while the target part 8 is irradiated with the adjusted amount of light, photocurrent generated in the photoelectric conversion unit 4 is measured and movements of the target part 8 are detected in accordance with changes in photocurrent (Steps S12 and S13). Detecting movements of the target part 8 after adjusting the amount of light will enable detection of movements of the target part 8 with a high degree of sensitivity irrespective of possible changes in the state in which the detection device 50 is attached or possible changes in the state of the target part 8.

The movements of the target part 8 are then counted (Step S14), and the number of counts is output to, for example, a monitor (Step S15).

The optimization processing and the counting and measurement processing, which have been described as separate flows, may be serial processing.

### Fifth Embodiment

A fifth embodiment relates to the configuration of the attachment portion 6. Description of the first to fourth embodiments holds true for the fourth embodiment as long as no inconsistencies are produced.

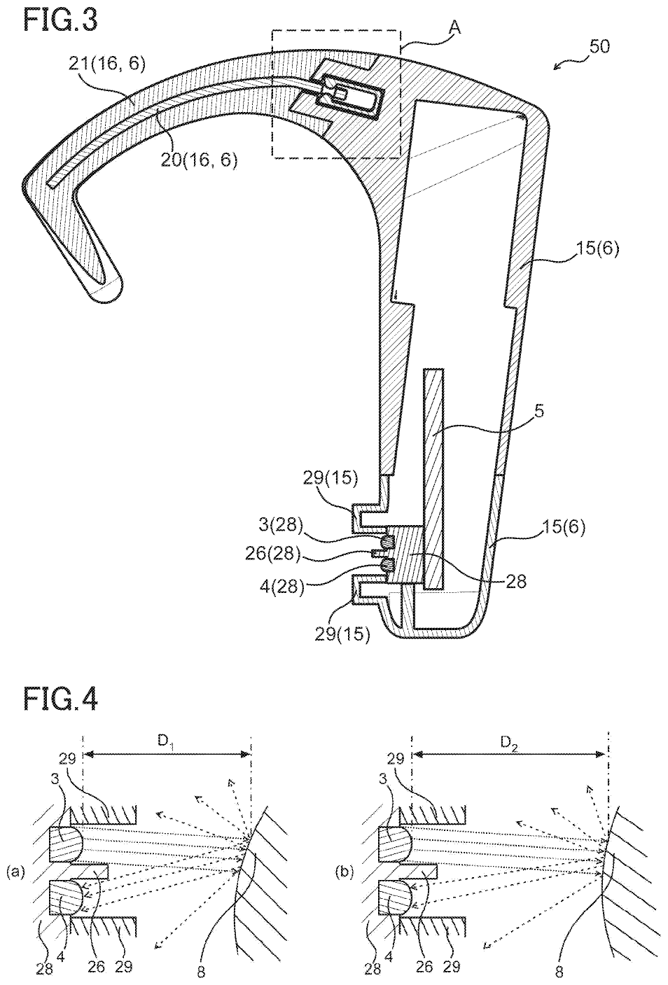

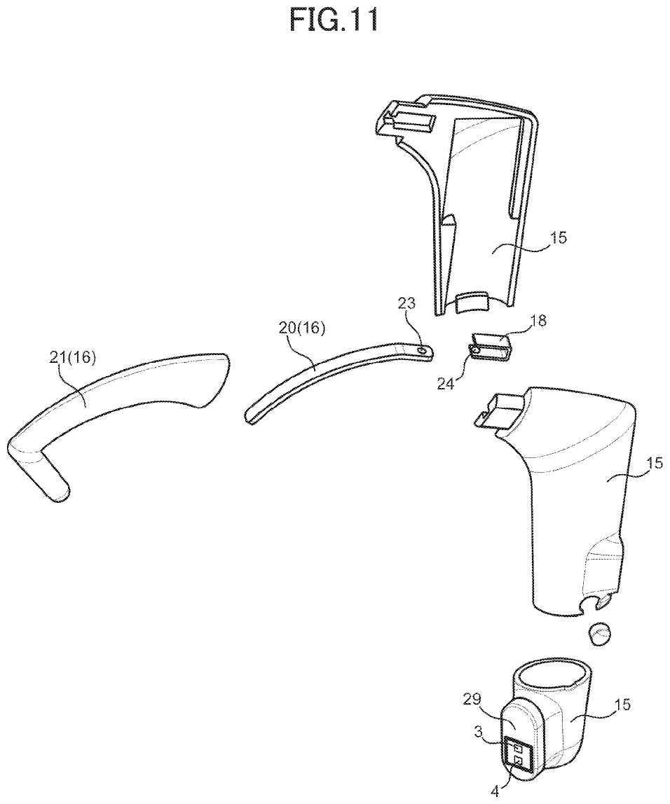

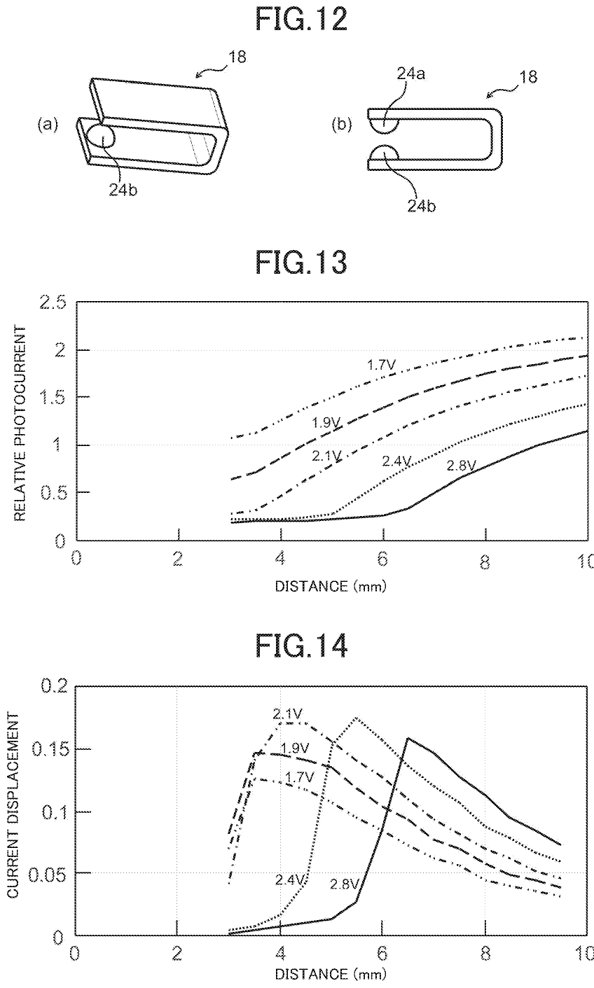

FIG. 3 is a schematic sectional view of the body-attachable detection device 50 according to the present embodiment. FIG. 10 is a partial sectional view of the detection device 50, illustrating a region A enclosed by the broken line in FIG. 3. FIG. 11 is a schematic exploded view of the detection device 50 according to the present embodiment. FIG. 12(a) is a schematic perspective view of a holder 18. FIG. 12(b) is a schematic side view of the holder 18.

In the present embodiment, the attachment portion 6 includes: the housing 15, which accommodates the light-emitting unit 3, the photoelectric conversion unit 4, and the control unit 5; the ear hook portion 16; and a holder 18 fixed to the housing 15 and made of metal.

The ear hook portion 16 includes a core portion 20 made of metal and a rubber portion 21, which covers the core portion 20. Since the ear hook portion 16 is configured as mentioned above, the user can wear the detection device 50 on the ear with a comfortable fit.

The ear hook portion 16 is removably fastened to the housing 15 in such a manner that a protruding portion 24 provided on one of the holder 18, which is fixed to the housing 15 and made of metal, and the core portion 20 of the ear hook portion 18 fits in a recessed portion or an opening 23 provided in the other one of the holder 18 and the core portion 20.

Since the ear hook portion 16 is removably fastened to the housing 15, the ear hook portion 16 may be replaced with another ear hook portion 16 conforming to the shape of the ear of the user. By selecting a fitted ear hook portion 16 and attaching it to the housing 15, the user can wear the detection device 50 on the ear with a comfortable fit.

Both the core portion 20 and the holder 18 are made of metal. Thus, the core portion 20 or the holder 18 may be elastically deformed so that the protruding portion 24 can fit in the recessed portion or in the opening 23 or the protruding portion 24 can be withdrawn from the recessed portion or from the opening 23. The ear hook portion 16 may be removably attached to the housing 15 accordingly.

Both the core portion 20 and the holder 18 are made of metal and are thus rigid, making the detection device 50 less prone to damage, which could be otherwise caused by attachment or removal of the ear hook portion 16. When the protruding portion 24 fits into the recessed portion or the opening 23, the user can feel a click and can thus recognize that the core portion 20 is in the right place in the holder 18. In this way, the user can enjoy enhanced usability.

As illustrated in FIGS. 3, 9, 10, and 11, the holder 18 may, for example, have a U-shaped section and may include two hemispherical protruding portions, namely, protruding portions 24a and 24b, which are provided on the inner surface forming the U-shape and face each other. The core portion 20 may have an end portion that can be inserted into the inner space defined by the U-shape of the holder 18. The end of the core portion 20 has the opening 23, into which the protruding portion 24a and 24b fit. Thus, the holder 18 can be elastically deformed when the end of the core portion 20 is inserted into the holder 18 and the core portion 20 is withdrawn from the holder 18. The ear hook portion 16 can be removably fastened to the housing 15 accordingly.

The core portion 20, which herein has the opening 23, may have a recessed portion in place of the opening 23. Alternatively, the core portion 20 may have a protruding portion and the holder 18 may have an opening or a recessed portion.

Photocurrent Measurement Experiment

A body-attachable detection device similar to the detection device illustrated in FIGS. 1 and 3 was prepared. As the light-emitting unit and the photoelectric conversion unit, a reflective photointerrupter (GP2S700HCP manufactured by Sharp Corporation) including an infrared light-emitting diode as the light-emitting unit and a pohtotransistor as the photoelectric conversion unit was used.

Voltages of 1.7 V, 1.9 V, 2.1 V, 2.4 V, and 2.8 V were applied across the light-emitting diode serving as the light-emitting unit, and the luminance of the light-emitting unit was varied accordingly. The luminance of the light-emitting diode increases with increasing magnitude of voltage applied across the light-emitting diode.

At each voltage level, the distance between the skin and each of the light-emitting unit and the photoelectric conversion unit was varied over a range of 3 to 10 mm (in increments of 0.5 mm), and photocurrent generated in the photoelectric conversion unit was measured. Results of measurements are illustrated in FIGS. 13 and 14. FIG. 13 illustrates how the relative photocurrent changed with varying distance, and FIG. 14 illustrates current displacement associated with the distance. A greater amount of current displacement denotes that movements of the skin were detected with a higher degree of sensitivity.

It was found that movements of the skin at a distance of 3 to 5 mm may be detected with a higher degree of sensitivity when the luminance of the light-emitting unit is relatively low. It was also found that movements of the skin at a distance of 5 to 10 mm may be detected with a higher degree of sensitivity when the luminance of the light-emitting unit is relatively high.

It is therefore understood that adjusting the luminance of the light-emitting unit in such a manner as to achieve a high degree of detection sensitivity prior to detection of skin movements will enable detection of skin movements with a high degree of sensitivity.

### REFERENCE SIGNS LIST

3 light-emitting unit
4 photoelectric conversion unit
5 control unit
6 attachment portion
8 target part
10 neutral-density filter
12 dimming structure
15 housing
16 ear hook portion
18 holder
20 core portion
21 rubber portion
23 opening
24, 24a, 24b protruding portion
26 partition
28 photointerrupter
29 spacer
50 body-attachable detection device

1. A body-attachable detection device comprising:
a light-emitting unit;
a photoelectric conversion unit;
a control unit; and
an attachment portion, wherein
the attachment portion is provided to attach the light-emitting unit, the photoelectric conversion unit, and the control unit to a living body,
the light-emitting unit is configured to project light onto a target part from which movements of the living body are to be detected,
the photoelectric conversion unit is configured to receive light reflected by the target part and to generate photocurrent, and
the control unit is configured to adjust, in accordance with the photocurrent generated in the photoelectric conversion unit, an amount of light to be projected onto the target part and to detect movements of the target part in accordance with changes in photocurrent that occur in the photoelectric conversion unit after the amount of light is adjusted.

2. The body-attachable detection device according to claim 1, wherein
the light-emitting unit includes a light-emitting diode, and
the control unit is configured to adjust the amount of light to be projected onto the target part by changing a magnitude of voltage applied across the light-emitting diode, by changing a magnitude of current flowing through the light-emitting diode, or by changing a period of time over which current flows through the light-emitting diode.

3. The body-attachable detection device according to claim 1, further comprising at least one neutral-density filter,
wherein the neutral-density filter and the control unit are provided in such a manner that the amount of light to be projected onto the target part is adjusted by inserting the neutral-density filter into a light projection path between the light-emitting unit and the target part and by replacing the neutral-density filter placed in the light projection path between the light emitting unit and the target part portion with another neutral-density filter.

4. The body-attachable detection device according to claim 1, further comprising a dimming structure placed in a light projection path between the light-emitting unit and the target part,
wherein the dimming structure and the control unit are provided in such a manner that the amount of light to be projected onto the target part is adjusted through the dimming structure.

5. The body-attachable detection device according to claim 1,
wherein the control unit is configured to:
determine changes in photocurrent that occur in the photoelectric conversion unit due to movements of the target part while the target part is irradiated with a first amount of light;
determine changes in photocurrent that occur in the photoelectric conversion unit due to movements of the target part while the target part is irradiated with light in an amount different from the first amount;
select, from the amounts of light projected onto the target part, the amount of light effecting the most significant changes in photocurrent; and
adjust the amount of light to be projected onto the target part so that the target part is irradiated with the selected amount of light.

6. The body-attachable detection device according to claim 1, wherein
the attachment portion includes: a housing that accommodates the light-emitting unit, the photoelectric conversion unit, and the control unit; an ear hook portion; and a holder fixed to the housing and made of metal,
the ear hook portion includes a core portion made of metal and a rubber portion that covers the core portion, and
the ear hook portion is removably fastened to the housing in such a manner that a protruding portion provided on one of the holder and the core portion fits in a recessed portion or an opening provided in the other one of the holder and the core portion.

7. The body-attachable detection device according to claim 6,
wherein the protruding portion is a hemispherical projection.
